(19) United States
(12) Patent Application Publication
(10) Pub. No.: US 2022/0110972 A1
(43) Pub. Date: Apr. 14, 2022

(54) APPARATUS AND METHOD FOR INACTIVATIVING VIRUSES AND PATHOGENS IN CONVALESCENT PLASMA UNITS FROM RECOVERED COVID-19 PATIENTS

(71) Applicant: Trevor Percival Castor, Arlington, MA (US)

(72) Inventor: Trevor Percival Castor, Arlington, MA (US)

(73) Assignee: Trevor P. Castor, Woburn, MA (US)

(21) Appl. No.: 17/499,792

(22) Filed: Oct. 12, 2021

Related U.S. Application Data
(60) Provisional application No. 63/090,711, filed on Oct. 12, 2020.

(51) Int. Cl. A61K 35/16; A61M 1/38

(52) U.S. Cl. CPC A61K 35/16; A61M 2205/3368; A61M 1/38

(57) ABSTRACT

The novel coronavirus COVID-19 has caused a worldwide pandemic of enormous proportions resulting in significant levels of morbidity and mortality, tremendous pressures on the healthcare system, personal freedoms and society, and an unprecedented impact on the economies of the United States and the world. There are still significant unknowns about this very contagious and deadly virus, and these unknowns are coupled with no natural immunity. A promising therapeutic strategy is the utilization/transfusion of convalescent plasma from recovered COVID-19 patients. There are, however, risks involved in such transfusions from residual virus and other adventitious viruses and bacteria. These risks can be minimized by the pathogen clearance of convalescent plasma units in a hospital setting. There is an immediate need for the rapid pathogen inactivation/clearance of convalescent plasma units from recovered COVID-19 patients.

The present invention is a physical pathogen reduction and inactivation apparatus and method for controlling or eliminating transfusion-transmittable infections in convalescent plasma from recovered COVID-19 donors. The invention inactivates both nonenveloped and enveloped viruses as well as pathogenic bacteria and parasites in units of human plasma, while retaining the potency of natural biologically-active proteinaceous products in the pathogen-reduced plasma. The invention uses critical, near-critical or supercritical fluids for viral and pathogen reduction of units of donor blood plasma in blood bags. The apparatus is in the form of a transportable mobile unit, where it can be used in hospitals, blood banks, and medical facilities.

### FIELD OF INVENTION

The present invention is directed to methods and apparatus for inactivating wide classes of viruses and other pathogens in units of blood plasma collected from recovered COVID-19 patients to prevent transfusion-transmitted infections. The process and apparatus feature critical, supercritical, or near critical fluids for inactivation of viruses and pathogens. The apparatus is preferably in the form of a portable, transportable, mobile unit.

### REFERENCES TO OTHER PATENTS

This application discloses a number of improvements and enhancements to the viral inactivation method and apparatus disclosed in U.S. Pat. No. 5,877,005 to Castor et al., which is hereby incorporated by reference in its entirety.

This application discloses a number of improvements and enhancements to viral inactivation method and apparatus disclosed in U.S. Pat. No. 6,465,168 to Castor et al., which is hereby incorporated by reference in its entirety.

This application discloses a number of improvements and enhancements to the method for inactivating viruses for use in vaccines as disclosed in U.S. Pat. No. 7,033,813 to Castor et al., which is hereby incorporated by reference in its entirety.

This application discloses a number of improvements and enhancements to the method for inactivating viruses as disclosed in published U.S. Patent Application No. 2006/0269928 to Castor, which is hereby incorporated by reference in its entirety.

This application is being filed simultaneously on the same date with related inventions as disclosed in U.S. Provisional Patent Applications Nos. 63/090,701, 63/090,707 and 63/090,713 to Castor, which are hereby incorporated by reference in their entirety.

### BACKGROUND OF THE INVENTION

The novel coronavirus COVID-19 has caused a worldwide pandemic of enormous proportions resulting in significant levels of morbidity and mortality, tremendous pressures on the healthcare system, personal freedoms and society, and an unprecedented impact on the economies of the United States and the world. There are still significant unknowns about this very contagious and deadly virus and these unknowns are coupled with no natural immunity, treatments, or vaccines.

The coronavirus, COVID-19, emerged in Wuhan, China in late December 2019. Coronaviruses are a large family of viruses that may cause illness in animals and humans. In humans, several coronaviruses are known to cause respiratory disease such as Middle East Respiratory Syndrome (MERS), Severe Acute Respiratory Syndrome (SARS) and the most recently discovered COVID-19. These viruses are all genetically related with both SARS (10% fatality rate) and MERS (37.4% fatality rate), being more deadly than COVID-19 but much less infectious. Should the next coronavirus be as infectious as COVID-19 but have a fatality rate approaching SARS or worse yet MERS, this future pandemic would be much more devastating than the current COVID-19 pandemic unless tools and capabilities are in place to contain and manage coronaviruses.

Industry and governments have responded to the current pandemic with an urgent search for new vaccines and for testing against COVID-19 a number of known antiretrovirals, previously developed for malaria, HIV, and Ebola.

One promising treatment is the transfusion of convalescent plasma from recovered COVID-19 patients into infected patients. Clinical trials are now being conducted on the use of convalescent plasma collected from recovered COVID-19 patients, and the FDA is facilitating access to COVID-19 convalescent plasma for use in patients with serious or immediately life-threatening COVID-19 infections through the process of single patient emergency Investigational New Drug Applications (eINDs) for individual patients under 21 CFR 312.310 [FDA, Mar. 5, 2020].

There are risks involved in such transfusions, caused by residual virus particles and other adventitious viruses and bacteria in the donor's plasma. According to the FDA, COVID-19 convalescent plasma must be collected from recovered individuals only if they are eligible to donate blood. The plasma must be tested and found suitable, and the convalescent donor may not have exhibited any symptoms at least 14 days prior to donation. However, there have been several reported cases showing that the COVID-19 virus can remain in circulation for periods much greater than 14 days after resolution of symptoms.

These risks can be minimized by the pathogen clearance of convalescent plasma units in a hospital setting. Ideally, convalescent plasma should be treated by a pathogen inactivation technology to ensure that the plasma is free of any residual SARS-CoV-2 and other adventitious pathogens. Current approaches such as pasteurization, solvent-detergent (SD), UV irradiation, and chemical and photochemical inactivation are not always effective against a wide spectrum of pathogens, are sometimes encumbered by process-specific deficiencies, and often result in denaturation of the biologics that they are designed to render safe. There are limited commercially available, FDA-approved technologies for the inactivation of viruses in units of human plasma.

There is an immediate need for the rapid pathogen inactivation/clearance of convalescent plasma units from recovered COVID-19 patients. The present invention is a method and apparatus, using the inventor's patented CFI™ pathogen inactivation technology that is capable of inactivating wide classes of viruses, bacteria, and parasites in units of COVID-19 convalescent plasma, with negligible negative impact on biological integrity and potency of the treated fluids. The pathogen activation technology could further be utilized in a hospital setting with a processing apparatus that is easily transportable to where it is needed.

### SUMMARY OF THE INVENTION

The present invention is a technology consisting of a method and apparatus for the rapid inactivation of coronaviruses and other pathogens in units of convalescent human plasma from recovered COVID-19 patients with minimal reduction in biological integrity and potency for the treatment of COVID-19 patients in a hospital setting. The technology, known as CFI™ (Critical Fluid Inactivation) uses critical, supercritical, or near critical fluids for inactivation of viruses and pathogens. The developed technology also has applicability to the cGMP manufacturing of anti-COVID-19 immunoglobulins from pools of plasma from COVID-19 recovered patients.

In one aspect of the present invention, the CFI™ pathogen inactivation technology operates, in part, by first permeating and inflating the virus particles with a selected Superfluid™ under pressure. The overfilled virus particles are then quickly decompressed, and the dense-phase fluid rapidly changes into a gaseous state, rupturing the virus particles at their weakest points. This is similar to the embolic disruption of the ear drums of a scuba diver who surfaces too rapidly. The disruption of the viral structures and release of nucleic acids prevents replication and infectivity of the CFI-treated viral particle.

The SuperFluids™ (SFS) of interest are normally gases, such as carbon dioxide and nitrous oxide, at room temperature and pressure. When compressed, these gases become dense-phase fluids, with enhanced thermodynamic properties of selection, solvation, penetration, and expansion. The ultra-low interfacial tension of SuperFluids™ allows facile penetration into nanoporous and microporous structures. As such, SFS can readily penetrate and inflate viral particles. Upon decompression, because of rapid phase conversion, the overfilled particles are ruptured and inactivated.

CFI technology, which inactivates both enveloped and non-enveloped viruses, is applicable to both pooled human plasma and units of plasma. The potential impact of a generally-applicable physical technology for inactivating both enveloped and non-enveloped viruses and emerging pathogens with high retention of biological activity is thus very significant. Such a technology, especially when used with conventional virus inactivation or pathogen reduction methods such as nanofiltration, will help ensure a blood supply that is safe from emerging and unknown pathogens and bioterrorism threats. In addition to human plasma and human plasma proteins such as fibrinogen and immunoglobulins, the developed technology will also be applicable to monoclonal antibodies and transgenic molecules.

The technology could be very impactful in developed countries and in hot zones for both the rapid virus clearance of pooled human plasma and units of plasma. The inventor developed two prototypes of this technology with versatility and cost efficiency that include: (i) an inexpensive bench-top prototype device that uses customized blood bags and can be readily deployed at community-level points-of-need where outbreaks occur, and (ii) pilot and large-scale CFI units to maximize high throughput processing at blood banks and hospitals, and industries (Industrial prototype). Both prototypes operate under similar CFI process conditions and use similar principles for pathogen inactivation. The technology offers unique advantages not achievable by currently available competing products like that of SD and the Cerus Intercept.

The present invention has advantages over the prior approaches that have been employed for the inactivation or removal of viruses in human plasma, harnessing therapeutic proteins derived from human plasma and preparation of recombinant biologics. These include heating or pasteurization; solvent-detergent technique; Ultra-Violet (UV) irradiation; chemical inactivation utilizing hydrolyzable compounds such as β-propiolactone and ozone; and photochemical decontamination using synthetic psoralens. The major problems with pasteurization include long pasteurization times, deactivation of plasma proteins and biologics, and the use of high concentrations of stabilizers that must be removed before therapeutic use. The solvent-detergent (SD) technique is quite effective against lipid-coated or enveloped viruses such as HIV, HBV and HCV, but is ineffective against protein-encased or non-enveloped viruses such as HAV and parvovirus B19. The solvent-detergent technique is also burdened by the need to remove residual organic solvents and detergents before therapeutic use. The photochemical-psoralen method, while quite effective with a wide range of viruses, is burdened by potential residual toxicity of photoreactive dyes and other potentially carcinogenic or teratogenic compounds.

The Cerus Intercept method has been shown to be effective against both enveloped and some but not all non-enveloped viruses. It has been recently approved by the FDA for the viral clearance of human plasma, red blood cells and platelets. HAV, HEV, B19, and Polio Virus are resistant to the Cerus inactivation process, but are sensitive to our CFI technology. Moreover, the Intercept method is restricted to units of plasma and is not applicable to pools of plasma, an advantage that the CFI offers since it was initially developed for pools of human plasma.

The major weakness of the Cerus Intercept process is that it requires removal of the reactive psoralen compounds which could be mutagenic and teratogenic. CFI does not require the removal of the reactive component that is readily removed by physical degassing over time and under vacuum. Additionally, CFI offers superiority in breadth in the number, types and strains of pathogens completely inactivated, with an accompanying simplicity, versatility and cost-efficiency. Thus, current approaches are not always effective against a wide spectrum of human and animal viruses, are sometimes encumbered by process-specific deficiencies, and often result in denaturation of the target biologics.

The prototypes developed for this technology have demonstrated versatility and cost-effectiveness: (i) an inexpensive bench-top prototype device that uses customized blood bags and can be readily deployed at community-level points-of-need where outbreaks occur, and (ii) pilot and large-scale CFI units to maximize high throughput processing at blood banks and hospitals, and industries (Industrial prototype). Both prototypes will operate under similar CFI process conditions and use similar principles for pathogen inactivation. The technology offers unique advantages not achievable by currently available competing products like that of SD and the Cerus Intercept.

These and other features, aspects and advantages of the present teachings will be better understood with reference to the following drawings, description, examples, and appended claims.

### BRIEF DESCRIPTION OF THE DRAWINGS

FIG. 1 shows a TEM (Transmission Electron Microscopy) photomicrograph of coronavirus;

FIG. 2 shows before-and-after TEM (Transmission Electron Microscopy) photomicrographs of normal viral activity before CFI and after CFI disruption and inactivation of bacteriophage D-6 virus;

FIG. 3 shows before-and-after SEM (Scanning Electron Microscopy) photomicrographs of normal viral activity before CFI and after CFI disruption and inactivation of yeast (Saccharomyces cerevisiae);

FIG. 4 is a schematic illustration of the CFIU bench-top unit process flow diagram; and

FIG. 5 an illustration of the CFIU bench-top unit, showing the side elevation and frame.

### DETAILED DESCRIPTION OF THE PREFERRED EMBODIMENTS

Coronaviruses constitute the subfamily Orthocoronavirinae, in the family Coronaviridae, order Nidovirales, and realm Riboviria. They are lipid-enveloped viruses with a positive-sense single-stranded RNA genome and a nucleocapsid of helical symmetry. The genome size of coronaviruses ranges from approximately 27 to 34 kilobases, the largest among known RNA viruses. The diameter of the virus particles is around 120 nm.

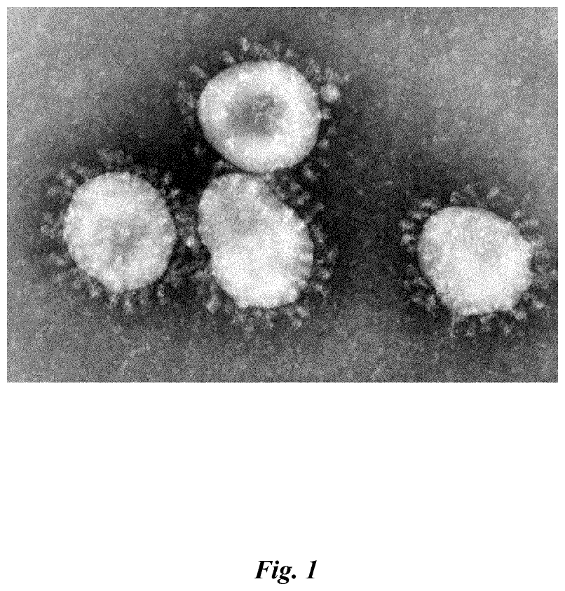

The envelope of the virus in electron micrographs appears as a distinct pair of electron dense shells, as shown in FIG. 1 with well-defined spikes. Infection begins when the virus enters the host organism and the spike protein attaches to its complementary host cell receptor. After attachment, a protease of the host cell cleaves and activates the receptor-attached spike protein. Depending on the host cell protease available, cleavage and activation allows cell entry through endocytosis or direct fusion of the viral envelop with the host membrane.

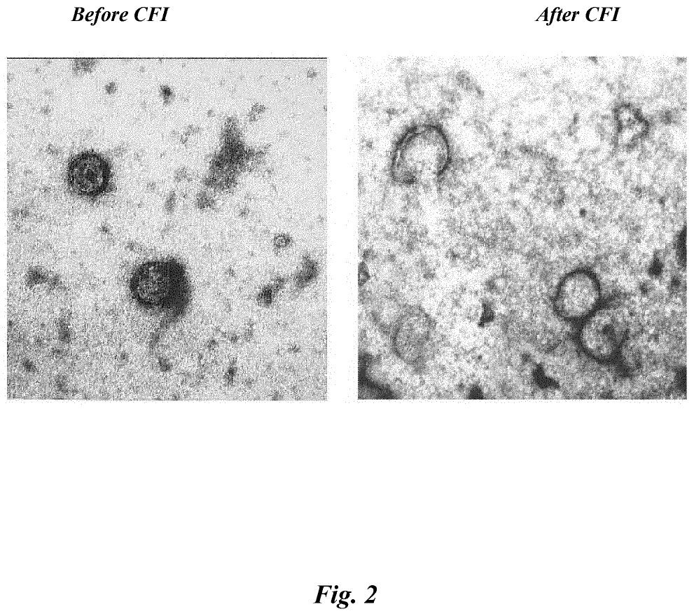

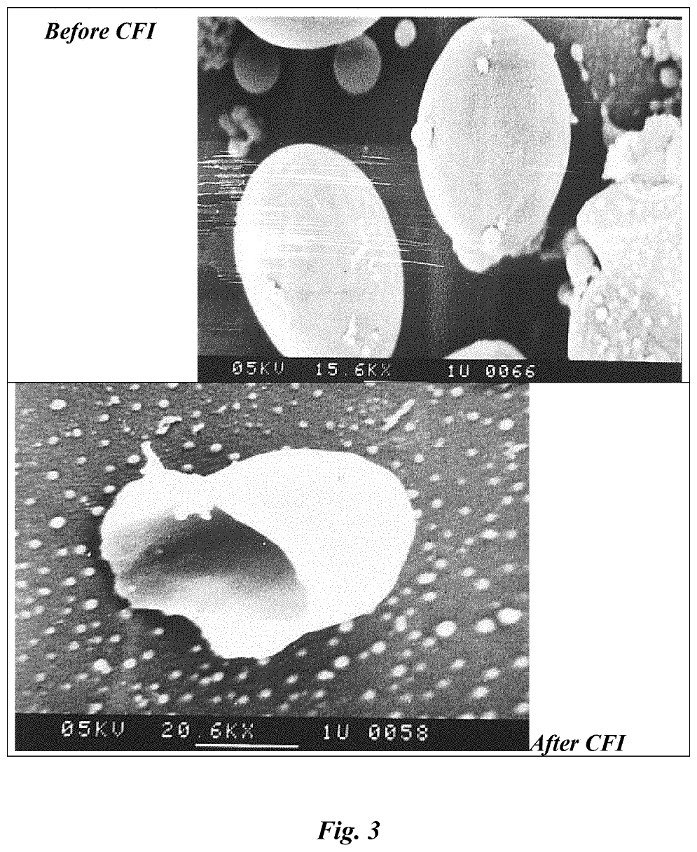

The CFI (Critical Fluid Inactivation) technology of the present invention has the capability to physically disrupt viral particles. FIG. 2 shows transmission electron microscopic (TEM) images of stains of bacteriophage virus <D-6 before and after CFI treatment. FIG. 3 shows before-and-after SEM (Scanning Electron Microscopy) photomicrographs of normal viral activity before CFI and after CFI disruption and inactivation of yeast (Saccharomyces cerevisiae). FIGS. 2 and 3 illustrate the ability of critical fluid inactivation (CFI) to inactivate enveloped viruses and a variety of other tough microorganisms. Also, like the SD technique developed by the New York Blood Center, CFI inactivates enveloped viruses by a lipid solubilization mechanism, dissolving away the protective lipid coat.

HCV 229E is able to grow in human cell lines such as MRC-5 and produces CPE consisting of rounding and sloughing of cells. MRC5 (ATCC CCL-171) is a human lung fibroblastic cell line obtained from a normal 14-week-old male fetus. It supports the replication of a number of respiratory viruses including human coronaviruses. MRC-5 cells, 80-90% confluent, will be infected at a relatively high multiple-of-infection (MOI of 0.1 to 0.2) and the virus will be harvested 24-48 hours post infection before CPE is visible. HCV OC43 shows no cross reactivity with HCV strain 229E, and is able to grow in human cell lines such as HCT-8 (ATCC CCL-244) and produces CPE consisting of vacuolation and sloughing of cells. Mouse hepatitis virus strain MHV-A59, a mouse coronavirus, will be grown in NCTC clone 1469, a mouse liver cell line, in which it produces CPE consisting of syncytia, rounding and sloughing of cells. Human coronavirus SARS-CoV-2 will be grown in human cell lines such as Vero (ATCC CCL-81) which produces CPE consisting of rounding and detachment of cells.

The virus stocks generated above were titrated in 96 well plates by our standard TCID₅₀ procedure on their respective host cells. Briefly, confluent monolayers of the host cells will be infected with serial log dilutions of the virus in replicates of 8. CPE will be monitored for 5-10 days and the number of wells showing CPE will be used to calculate the TCID₅₀ by the Karber method. The duration of the assay that gives the highest titers will be optimized initially. Additionally, virus titrations will also be performed by qPCR of viral nucleic acids and ELISA and/or lateral flow assays for viral antigens in the culture supernatants in the TCID₅₀ assay.

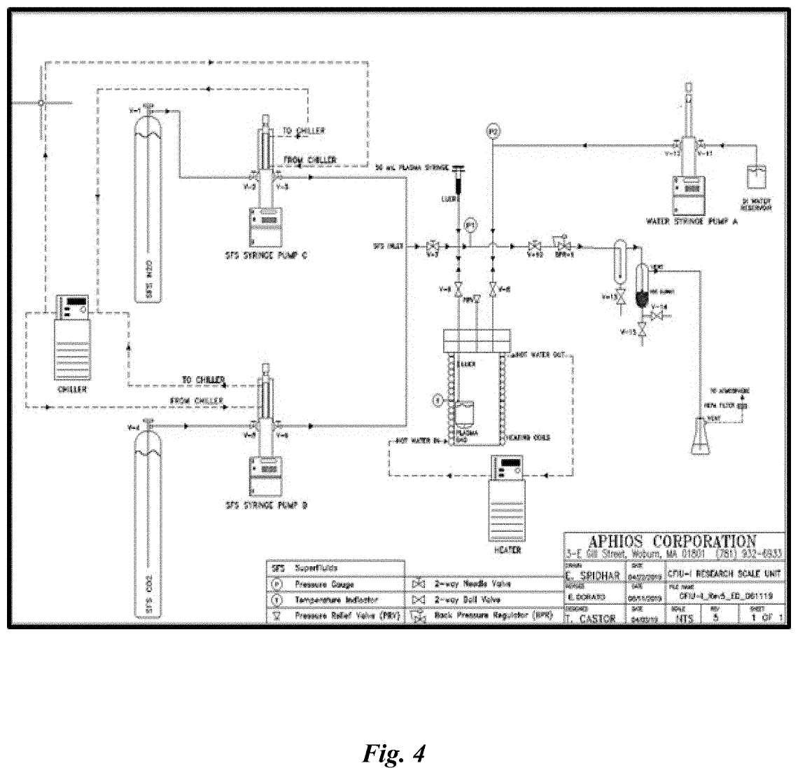

CFI experiments are conducted in a CFIU prototype, as shown in FIG. 4, which is a bench-top unit, designed to be easily transportable to the point of use, such as a hospital serology laboratory. The center of the CFIU prototype is a 316 stainless-steel (SS) high-pressure cylinder (9.5 cm OD×33.5 cm L) with an internal volume of approx. 800 mL containing a plasma bag on a Luer-Lok attached to a ⅛″ 316 SS high-pressure tubing rated at 8,500 psig. This tubing passes through a top flange and cap, and is secured in place with ⅛″ Swagelok fittings. The cap is secured to the vessel with 8¾″ screws torqued to 60 ft-lb. The vessel is rated for 5,000 psig at 22° C. with a hydrostatic test pressure of 6,500 psig (High Pressure Equipment Company, Erie, Pa., USA). The vessel is equipped with a pressure relief valve (PRV) set at 3,000 psig, the planned operating pressure of the prototype. The vessel is wrapped with copper heating coils originating from a circulation heater in order to maintain the vessel at operating temperature, usually ˜ 40° C.

Plasma is first introduced into the plasma bag via a 50 mL syringe attached to the ⅛″ SS tubing outside of the cylinder via a second Luer-Lok. Alternatively, as planned for commercial units, the plasma bag with plasma is connected to the first Luer-Lok and the vessel sealed before proceeding to the next operational step. After the plasma is introduced, valve V-9 is closed. All three high-pressure Isco syringe pumps are then zeroed. Warm water (˜40° C.) is then introduced into the vessel via water syringe pump C by opening V-8 and V-12 connected to the water syringe pump C. Fresh water is resupplied to the water syringe pump C via valve V-11. After coming to operating temperature, the system is pressurized to operating pressure.

The plasma is then kept at operating pressure and temperature for a specific residence time of minutes to an hour. After the designated residence time has been achieved, the plasma bag is decompressed by first opening valve V-10 and releasing pressure through the back-pressure regulator, BPR-1. The effluent SFS, now a gas, is bubbled through a liquid trap containing 10% Chlorox and then vented through a HEPA filter to the atmosphere. Simultaneous to the decompression of the plasma bag, the pressure outside of the plasma bag is reduced by running pump C in reverse. The treated plasma is then recovered.

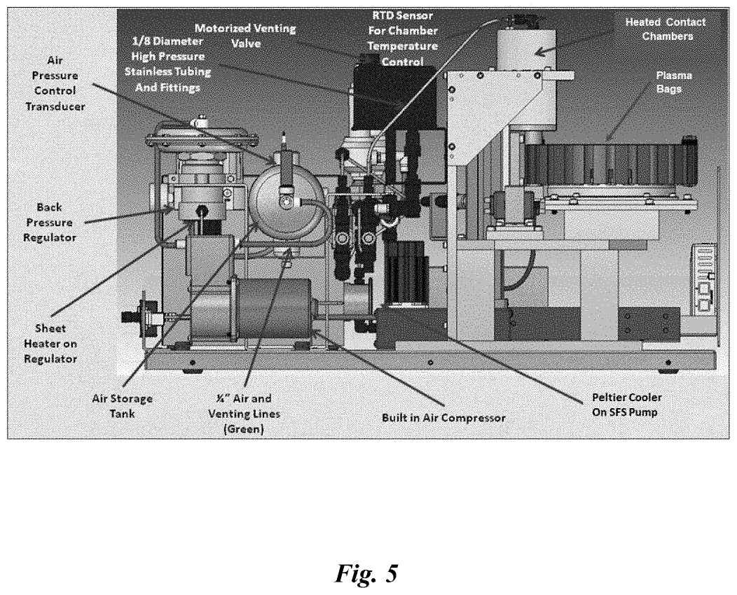

FIG. 5 shows a design for a beta-site CIFU unit for treating COVID-19 convalescent plasma in a hospital setting. The design in FIG. 5 has the capability of processing several plasma units sequentially, using a rotating carousel design. One operation procedure is performed on each plasma unit in sequence. Once a plasma bag is processed, the plasma bag will be disengaged from the SFS feed and pressurizing source, and the carousel will be rotated, advancing the CFI-processed plasma bag for automatic removal. Subsequently, a new plasma bag will be rotated into place for processing.

The detailed description set forth above is provided to aid those skilled in the art in practicing the present invention. However, the invention described and claimed herein is not limited in scope by the specific embodiments herein disclosed. The embodiments are intended as illustrations of several aspects of the invention. Any equivalent embodiments are intended to be within the scope of this invention. Various modifications of the invention in addition to those shown and described herein will become apparent to those skilled in the art from the foregoing description which do not depart from the spirit or scope of the present inventive discovery. Such modifications are also intended to fall within the scope of the appended claims.

### EXAMPLES

### Example 1: Single-Stage CFIU™ Inactivation of Human Coronavirus-229E (HCV-229E) Virus Using 1% CO₂ in a SFS Mixture N₂O:CO₂::99:1 (CFIU-II-180)

In this single-stage CFIU-II-180 experiment, FBS was used instead of human plasma to avoid neutralization of human viruses by potential antibodies in donor plasma. The sample bag was loaded with 80 mL of SFS (N₂O:CO₂::99:1 at ˜2,250 psig and 40° C.) followed by −60 mL of FBS sample.

The virus titration results are listed in Table 2. The spike control showed a titer of 4.94 log TCID₅₀/mL, and the 4° C. control had a titer of 3.87 log TCID₅₀/mL, consistent with spiking the plasma with the virus stock at a 1:10 ratio. The Virus Reduction Factor (VRF) obtained for the CFI bag product before degassing was 1.61 log TCID₅₀.

TABLE 2
CFIU-II-180 - HCV-229E Virus Titration
Results (6 days Post Infection)

| CFIU-II-180 | Dilution | Number of | Titer (log | VRF (log |
|---|---|---|---|---|
| Sample | (3^{u}) | wells +/8 | TCID₅₀/mL) | TCID₅₀) |
| 4° C. Plasma | 3 | 8 | 3.87 | 0.00 |
| Control | 4 | 7 | | |
| | 5 | 2 | | |
| | 8 | 1 | | |
| | 3 | 8 | 3.87 | 0.00 |
| t&T Plasma | 4 | 5 | | |
| Control | 5 | 2 | | |
| | 6 | 3 | | |
| CFIU Bag | 0 | 6 | 2.26 | 1.61 |
| Product | 1 | 8 | | |
| | 2 | 1 | | |
| | 3 | 0 | | |
| Spike Control | 5 | 8 | 4.94 | N/A |
| | 6 | 7 | | |
| | 7 | 4 | | |
| | 8 | 1 | | |

Inactivation levels can be increased by increasing the number of transfers of plasma to SFS or stages as demonstrated in U.S. Provisional Patent Application No. 63/090,707.

### Example 2: Characterization of CFI Treated Human Plasma

Changes to the composition, characteristics and functionality of the components of CFI treated plasma are identified to determine if these treatments result in changes that would lead to actual or potential adverse events upon transfusion of the respective treated plasma to recipients. The changes from the following treatments will be monitored and compared with untreated plasma. We compare: (1) physicochemical characteristics including protein aggregation and clot strength assays; (2) protein profiles by denaturing and native electrophoresis (1-D and 2-D); (3) secondary modifications of proteins (glycosylation, acylation, phosphorylation); (4) functionality of coagulation factors by assays such as PT, APTT, TT and specific single factor assays for intrinsic and extrinsic pathways; (5) vWF assays by ELISA, collagen binding assay and RIPA assay; (6) quantification by ELISA of various proteins in the clotting cascade such as fibrinogen, etc.; and (7) biochemical assays by SMAC analysis, a panel of 24 clinical laboratory parameters.

### Example 3: Evaluate Antibodies to SARS-CoV-2 for Biological Activity and Potency as Well as Other Key Plasma Transfusion Biological Properties by In Vitro Immunological Assays

We evaluate the biological and activity of convalescent COVI-19 plasma treated by CFI technology for pathogen inactivation by in vitro immunological and biochemical assays. We have previously demonstrated that CFI technology has little of no impact on the bioactivity and potency of immunoglobulins. The effects of CFI N₂O on a hyper-immunoglobulin at different temperatures (22 to 40° C.) and pressures (0 to 278 bars) are listed in Table 2 and compared to controls at atmospheric pressure showing little or no change in physical and potency parameters tested. We have also demonstrated that antigenicity and immunogenicity of HIV is retained after CFI inactivation.

TABLE 2
Effect of CFI N2O at Different Pressures
and Temperatures on a Hyperimmuneglobulin

| NO₂ | HPLC- | Anti- | Protein | ELISA |
|---|---|---|---|---|
| bars/° C. | SEC (%) | Complementary | (mg/ml) | MEP Abs |
| 0/22 | 94.7 | >1.74 | 18.14 | 379.5 |
| 278/22 | 95.2 | >1.74 | 17.39 | 370.8 |
| 0/29 | 101.4 | >1.83 | 18.27 | 349.7 |
| 208/29 | 92.7 | >1.77 | 17.65 | 313.8 |
| 0/40 | 104.3 | >1.81 | 18.00 | 351.4 |
| 278/40 | 99.7 | >1.78 | 17.84 | 385.4 |

### Example 4: Virus Neutralization Assay for Antibody Activity and Potency

Virus neutralization assays are performed using the widely used and accepted classical infectivity titration assays. This assay is considered as the definitive proof in vitro of the functionality of an antibody preparation. In this assay, serial dilutions of the plasma/antibody are incubated in the presence of 100 TCID₅₀ of the virus at 37 C for 1 hour and then added to the host cell monolayers in replicates of 8 in 96 well plates. The cells are monitored for CPE for the required number of days needed for highest sensitivity and the neutralization titers are calculated by the Karber method as the dilution at which CPE is inhibited in 50% of the time. Alternately, qPCR for viral RNA is performed on the culture supernatants at earlier time points to shorten the duration of the assay using published methods. The neutralization titers of the CFI treated plasma preparations are compared with those of the untreated counterparts.

### Example 5: ELISA for Preservation of Antibody Titers

The CFI treated plasma samples are tested for the preservation of antibody titers by ELISA against S glycoprotein of the virus. Although patients can develop antibodies against multiple viral antigens, the focus is on the antibodies to S glycoprotein since these antibodies have the ability to prevent the binding of the virus to the host cell receptor thereby eliminating infectivity of the virus. ELISAs are performed against both the full-length S glycoprotein and the Receptor Binding Domain (RBD) by published methods (Amanat et al, 2020). In addition to detection of IgG, we also test the samples using secondary antibodies to IgM and IgA and correlate the results with neutralization titers to determine the role that these antibody classes play in protection and confirm that the CFI treatment preserves these activities as well. Additional methods such as western blotting are used, if necessary. CFI treated convalescent COVID-19 plasma with an antibody integrity and potency at least 90% of untreated convalescent COVID-19 plasma is recommended.

Thus, various embodiments of the present technology been described, and said embodiments are capable of further modification and variation by those skilled in the art. Accordingly, it is intended that the examples and the description be intended for illustration purposes only and that the inventions set forth in the claims shall encompass variations and equivalents.

What is claimed is:

1. A treatment for COVID-19 patients using convalescent plasma which is pathogen reduced by SuperFluids which are supercritical, near-critical and critical fluids with or without small molar quantities of polar cosolvents.

2. The treatment of claim 1 wherein the SuperFluids are nitrous oxide (N₂O) and carbon dioxide (CO₂).

3. The treatment of claim 2 wherein the ratio of N₂O to CO₂ ranges from 90% to 100% N₂O, and from 10% to 0% CO₂.

4. The treatment of claim 3 wherein the ratio of N₂O to CO₂ 99% N₂O to 1% CO₂

5. The treatment of claim 2 wherein the SuperFluids are at a pressure of 2,000 to 5,000 psig and a temperature of 20° C. to 50° C.

6. The treatment of claim 6 wherein the SuperFluids are at a pressure of 2,500 to 3,500 psig and a temperature of 35 to 40° C.

7. The treatment of claim 6 wherein the SuperFluids are at a pressure of 3,000 psig and a temperature of 37° C.

8. A method of treating for COVID-19 patients using convalescent plasma which is pathogen reduced by SuperFluids which are supercritical, near-critical and critical fluids with or without small molar quantities of polar cosolvents.

9. The method of claim 8 wherein the SuperFluids are nitrous oxide (N₂O) and carbon dioxide (CO₂).

10. The method of claim 9 wherein the ratio of N₂O to CO₂ ranges from 90% to 100% N₂O, and from 10% to 0% CO₂.

11. The method of claim 10 wherein the ratio of N₂O to CO₂ 99% N₂O to 1% CO₂

12. The method of claim 9 wherein the SuperFluids are at a pressure of 2,000 to 5,000 psig and a temperature of 20° C. to 50° C.

13. The method of claim 12 wherein the SuperFluids are at a pressure of 2,500 to 3,500 psig and a temperature of 35 to 40° C.

14. The method of claim 13 wherein the SuperFluids are at a pressure of 3,000 psig and a temperature of 37° C.

15. An apparatus for making multiple units of pathogen-reduced COVID-19 convalescent plasma which is pathogen reduced by SuperFluids which are supercritical, near-critical and critical fluids with or without small molar quantities of polar cosolvents.

16. The apparatus of claim 15 which comprises:
(a) a pressure vessel containing plasma in a sample bag surrounded by a hydraulic fluid;
(b) a pump for increasing or decreasing the volume or pressure of the hydraulic fluids surrounding the sample bag;
(c) a pressure vessel containing SuperFluids in a product bag surrounded by a hydraulic fluid;
(d) a pump for increasing or decreasing the volume or pressure of the hydraulic fluids surrounding the product bag;
(e) a pump for introducing a SuperFluids into the product bag;
(f) a pump for introducing a second SuperFluids into the product bag;
(g) chillers for maintaining the SuperFluids in a liquid state;
(h) heaters for maintain the temperature of the hydraulic fluids in the pressure vessels;
(i) connecting lines to move fluids from the sample bag to the product bag;
(j) a back-pressure regulator to contain and release pressure in the apparatus;
(k) controllers for managing volumes, pressures and temperatures; and
(l) a rotating carousel for processing several plasma units sequentially, once a plasma bag is processed, the plasma bag will be disengaged from the SFS feed and pressurizing source, and the carousel will be rotated, advancing the CFI-processed plasma bag for automatic removal. Subsequently, a new plasma bag will be rotated into place for processing.

17. The apparatus of claim 16 wherein the hydraulic fluid is oil or water.

18. The apparatus of claim 17 wherein the hydraulic fluid is water.

19. The apparatus of claim 16 wherein the sample and product bags are multiport plastic bags.

20. The apparatus of claim 19 wherein the multiport plastic bags are made of polyvinyl chloride (PVC), polytetrafluoroethylene (PTFE), perfluoroalkoxy alkanes (PFA) or fluorinated ethylene propylene (FEP).
